(19) United States
(12) Patent Application Publication
(10) Pub. No.: US 2025/0025702 A1
(43) Pub. Date: Jan. 23, 2025

(54) IMPLANTABLE MEDICAL DEVICE CHARGER APPARATUS WITH WEARABLE CHARGER SUPPORTS

(71) Applicant: The Alfred E. Mann Foundation for Scientific Research, Valencia, CA (US)

(72) Inventors: Susanna M. Lin, Los Angeles, CA (US); Joseph L. Calderon, Santa Clarita, CA (US); Edward Hillery, Valencia, CA (US)

(73) Assignee: The Alfred E. Mann Foundation for Scientific Research, Valencia, CA (US)

(21) Appl. No.: 18/740,465

(22) Filed: Jun. 11, 2024

Related U.S. Application Data
(60) Provisional application No. 63/514,730, filed on Jul. 20, 2023.

(51) Int. Cl. A61N 1/378; A61N 1/36; A61N 1/372

(52) U.S. Cl. CPC A61N 1/3787; A61N 1/36125; A61N 1/37229

(57) ABSTRACT

An implantable medical device (IMD) charger apparatus includes an IMD charger with a transmission component, an electronics component and a cable that extends from the transmission component to the electronics component, and a wearable IMD charger support including a first chest part, a second chest and a neck part that extends from the first chest part to the second chest part, and a cable holder associated with the neck part.

### CROSS-REFERENCE TO RELATED APPLICATION

This application claims the benefit of U.S. Provisional Application No. 63/514,730, filed Jul. 20, 2023, and entitled “Medical Device Charger Apparatus With Wearable Charger Supports,” which is incorporated herein by reference.

### BACKGROUND OF THE INVENTIONS

### 1. Field of the Inventions

The present inventions relate generally to the charging of rechargeable power sources in implanted medical devices.

### 2. Description of the Related Art

Implantable medical devices (“IMDs”) such as, for example, implantable pulse generators, vagus nerve stimulators and drug infusion pumps, frequently include therapeutic and/or diagnostic components as well as rechargeable power sources such as rechargeable batteries, capacitors, and combinations thereof. External IMD chargers (or “IMD chargers”) are used to wirelessly and transcutaneously transmit power to IMDs via inductive coupling. To that end, IMD chargers include a primary charging coil and the IMDs include a secondary charging coil. The primary charging coils of the IMD chargers may be energized to create a magnetic charging field that induces current in the secondary charging coils of the IMDs. The current is used to recharge the batteries or other rechargeable power sources.

One issue associated with the use of IMDs and IMD chargers is the distance between the primary and secondary charging coils, which should be minimized to the extent practicable. Another issue is charging alignment. Each charging coil defines a central axis and the coils are aligned when the axes are colinear. Mis-alignment of the charging coils reduces the efficiency of the power transfer and, accordingly, some IMD chargers are configured to determine whether or not the primary and secondary charging coils are properly aligned with one another and to provide feedback, such as audible, visible and/or tactile feedback, that is indicative of whether or not the charging coils are properly aligned. Examples of IMD chargers with alignment determination and audible, visible and/or tactile feedback capabilities may be found in, for example, US Pat. Pub. No. 2013/0096651 A1 and US Pat. Pub. No. 2017/0361115 A1, which are incorporated herein by reference.

In some instances, a garment or other wearable article (collectively “wearable articles”) may be used to maintain the aligned position of the IMD charger on the body relative to the IMD. The IMD charger and the wearable article in some instances include respective hook and loop portions of a hook and loop fastener (commonly sold under the tradename Velcro) that secures the IMD charger to the wearable article when the IMD charger is pressed against the wearable article. Examples of such IMD chargers and wearable articles may be found in, for example, US Pat. Pub. No. 2013/0096651 A1.

### SUMMARY

The present inventors have determined that conventional IMD chargers and the manner in which the primary charging coil is aligned with the secondary charging coil of the associated IMDs, as well as the manner in which the IMD chargers are secured to the IMD recipient, are susceptible to improvement. For example, one proposed IMD charger includes an electronics component that is connected to a transmission component by a cable. The electronics component may include a controller and a power source, while the transmission component may include a primary charging coil. The transmission component of the IMD charger is positioned by way of a wearable article in such a manner that the primary and secondary charging coils are aligned. The present inventors have determined that conventional wearable articles for use with IMD chargers are susceptible to improvement. In particular, in some instances where the IMD charger includes a cable, it has been proposed that the cable extend around the IMD recipient's neck. The present inventors have determined that such cable positioning can result in the cable becoming entangled with hair or jewelry and/or rubbing against the IMD recipient's neck and causing discomfort.

An IMD charger apparatus in accordance with at least one of the present inventions may include an IMD charger with a transmission component, an electronics component and a cable that extends from the transmission component to the electronics component, and a wearable IMD charger support including a first chest part, a second chest, a neck part that extends from the first chest part to the second chest part, and a cable holder associated with the neck part.

A wearable IMD charger support in accordance with at least one of the present inventions may include a first chest part with a first chest part fastener that is configured to mate with an IMD charger transmission component fastener and/or an IMD charger electronics component fastener, a second chest part with a second chest part fastener that is configured to mate with an IMD charger transmission component fastener and/or an IMD charger electronics component fastener, a neck part that extends from the first chest part to the second chest part, and means for covering the cable and holding the cable against the neck part.

A wearable IMD charger support in accordance with at least one of the present inventions may include a first chest part with a first chest part fastener that is configured to mate with an IMD charger transmission component fastener and/or an IMD charger electronics component fastener, a second chest part with a second chest part fastener that is configured to mate with an IMD charger transmission component fastener and/or an IMD charger electronics component fastener, a neck part that extends from the first chest part to the second chest part and includes a neck part fastener, and a cable holder including a flexible panel, with a first edge that is permanently secured to the neck part and a second edge that is not permanently secured to the neck part, and at least one panel fastener associated with the second edge that is configured to mate with the neck part fastener.

There are a number of advantages associated with the present IMD charger apparatus and wearable IMD charger supports. By way of example, but not limitation, the cable holder covers the cable and prevents the charger cable from rubbing against the recipient's neck, thereby preventing the charger cable from becoming entangled with hair or jewelry and rubbing against the IMD recipient's neck.

### BRIEF DESCRIPTION OF THE DRAWINGS

Detailed descriptions of exemplary embodiments will be made with reference to the accompanying drawings.

FIG. 1 is a front view of an implantable medical device (IMD) charger apparatus, including an IMD charger and a wearable IMD charger support, in accordance with one embodiment of a present invention.

FIG. 2 is a front view of the IMD charger illustrated in FIG. 1.

FIG. 3 is a rear view of the IMD charger illustrated in FIG. 1.

FIG. 4 is a section view of a portion of the IMD charger illustrated in FIG. 1.

FIG. 5 is a section view of a portion of the IMD charger illustrated in FIG. 1.

FIG. 6 is a block diagram of the IMD charger apparatus illustrated in FIG. 1 in combination with an exemplary IMD.

FIG. 7 is a perspective view of the wearable IMD charger support illustrated in FIG. 1.

FIG. 8 is a front view of the wearable IMD charger support illustrated in FIG. 1.

FIG. 9 is a rear view of the wearable IMD charger support illustrated in FIG. 1.

FIG. 10 is an exploded view of the wearable IMD charger support illustrated in FIG. 1.

FIG. 11 is a section view taken along line 11-11 in FIG. 8.

FIG. 12 is a rear view of a portion of the wearable IMD charger support illustrated in FIG. 1.

FIG. 13 is a partially exploded view of a portion of the wearable IMD charger support illustrated in FIG. 1.

FIG. 14 is a perspective view of a portion of the IMD charger apparatus illustrated in FIG. 1 with the cable holder in an open state.

FIG. 15 is a side view of a portion of the wearable IMD charger support illustrated in FIG. 1.

FIG. 16 is a front view of a wearable IMD charger support in accordance with one embodiment of a present invention.

FIG. 17 is a front view of a wearable IMD charger support in accordance with one embodiment of a present invention.

FIG. 18 is a front view of a wearable IMD charger support in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

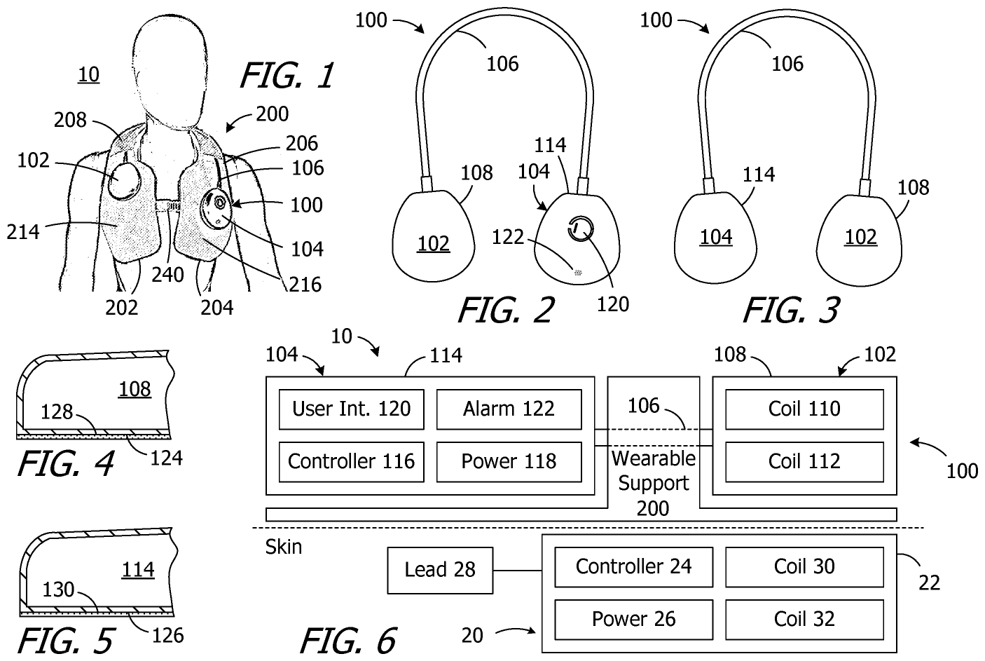

As illustrated for example in FIGS. 1 and 6, an implantable medical device (IMD) charger apparatus 10 in accordance with one embodiment of a present invention may be used in conjunction with an IMD 20. Although the present inventions may be used in conjunction with a wide variety of IMDs, the exemplary IMD 20 (e.g., an implantable pulse generator) includes a case 22 formed from a biocompatible material such as titanium, a controller 24, a battery or other rechargeable power source 26, and a lead 28 with a plurality of conductive contacts (not shown). Power and data transmission may be facilitated by a secondary charging coil 30, a telemetry coil 32. and associated circuitry (not shown). The charger apparatus 10 includes an IMD charger 100 that is configured for use with the IMD 20 and a wearable IMD charger support (or “wearable charger support”) 200 that positions the appropriate portion of the IMD charger on the recipient's chest and over the corresponding portion of the IMD.

The exemplary IMD charger 100 illustrated in FIGS. 1-6 includes a transmission component 102, an electronics component 104, and a cable 106 that electrically and mechanically connects the transmission component to the electronics component. The transmission component 102 may include a housing 108, a primary charging coil 110 for power transmission and a telemetry coil 112 for data transmission. Power and data transmission occurs transcutaneously through a recipient's skin and other tissue by way of inductive coupling between the IMD secondary charging coil 30 and a telemetry coil 32 and the IMD charger primary charging coil 110 and telemetry coil 112. The electronics component 104 may include a housing 114, a controller 116, a power source 118 (either rechargeable or replaceable), a user interface 120 (e.g., a power button) and an alarm 122 that is configured to provide an audible, visible and/or tactile indication that, for example, the IMD primary charging coil 110 is aligned with IMD secondary charging coil 30, as determined by the controller 116. The length of the cable 106 is such that the transmission component 102 and electronics component 104 may be located on opposite sides of the recipient's chest as shown in FIG. 1. The length of the cable 106 may, for example, range from about 10 inches to 30 about inches and is about 21 inches in the illustrated embodiment. As used here in the context of dimensions, the word “about” means +10%.

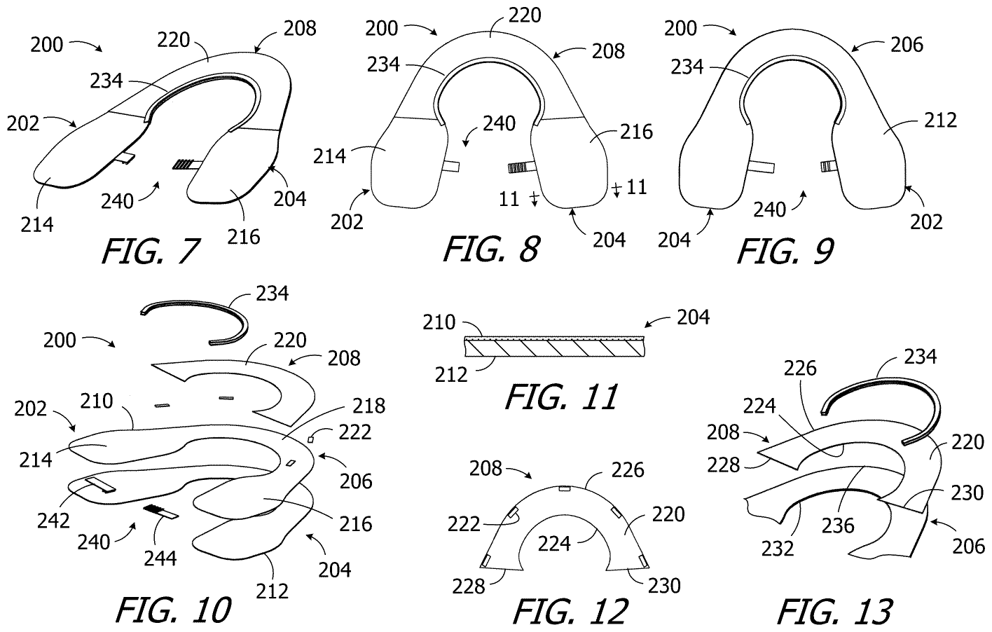

The transmission component 102 and an electronics component 104 may be configured to be fastened to the wearable charger support 200 in the manner described in greater detail below with reference to FIGS. 7-14. Although the present inventions are not limited to any particular attachment instrumentalities, fasteners may be employed and the exemplary implementation employs a hook and loop fastener arrangement. Referring more specifically to FIGS. 4 and 5, which show the transmission component and electronics component housings 108 and 114 without the associated internal elements, the housing bottoms 124 and 126 include hook portions 128 and 130 of a hook and loop fastener. As used herein, the words “bottom” and “rear” refer to the portion of the IMD charger apparatus 10 that faces the recipient when in use, and the words “top” and “front” refer to the portion of the IMD charger apparatus that faces away from the recipient when in use. The hook material that forms the hook portions 128 and 130 may be secured to the housings 108 and 114 with adhesive. The hook portions 128 and 130 may be secured to corresponding chest part fasteners 214 and 216 (FIG. 1) on the wearable charger support 200 which, as discussed in greater detail below, are loop portions of a hook and loop fastener.

Turning to FIGS. 7-14, the exemplary wearable charger support 200 includes a first chest part 202, a second chest part 204, a neck part 206 that extends from the first chest part to the second chest part, and a cable holder 208 associated with the neck part. In the illustrated embodiment, the first chest part 202, the second chest part 204 and the neck part 206 are portions of an integral structure formed from a top panel 210 and a bottom panel 212. The top and bottom panels 210 and 212 may be secured to one another by, for example, stitching around the perimeter thereof. Binding material, which is folded over the stitching and sewn or glued in place, may also be used to cover the stitching in some instances. The first and second chest parts 202 and 204 also respectively include chest part fasteners that are configured to mate with fasteners 128 and 130 on the IMD charger transmission component 102 and electronics component 104. In the illustrated embodiment, the top panel 210 is a fabric panel that forms the loop portion of a hook and loop fastener. The chest part fasteners 214 and 216 are defined by areas of the top panel 210 that are within the first and second chest parts 202 and 204, while a neck part fastener 218 is defined by the top panel area within the neck part 206. The bottom panel 212 may be formed from a material that provides comfort and stability and may also facilitate heat and sweat dissipation. Suitable bottom panel materials include, but are not limited to, neoprene spandex, polyester or nylon mesh, and grip fabric.

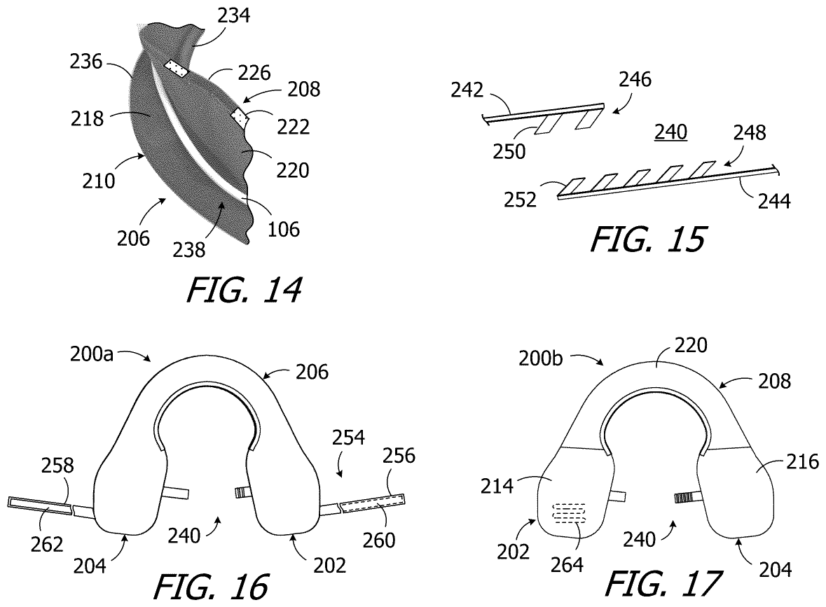

The exemplary cable holder 208, which may be used to cover a cable (e.g., cable 106) and holds the cable against the neck part 206, includes a flexible panel 220 and one or more fasteners 222. The panel 220 is secured to the neck part 206 in such a manner that the cable holder 208 can be moved between a closed state (FIGS. 1 and 7) where the cable holder will cover the cable and an open state (FIG. 14) where the cable can be moved into position between the neck part 206 and the cable holder or removed from this position. In the exemplary embodiment, the perimeter of the panel 220 (and cable holder 208) is defined by an inner edge 224, an outer edge 226 and end edges 228 and 230 that face the chest parts 202 and 204. The portion of the cable holder panel 220 at and adjacent to the inner edge 224 is permanently attached the neck part 206 at and adjacent to the neck panel inner edge 232 by, for example, binding tape 234 or any other suitable instrumentality. As used herein, a “permanently attached” cable holder panel is a cable holder panel that is attached to the neck part in such a manner that removal of the cable holder panel from the neck part involves damage to the cable holder panel, the neck part, and/or the instrumentality holding the two together. The remainder of the cable holder panel 220 is not permanently attached to the neck part 206 and, instead, the fasteners 222 that are adjacent to the outer edge 236 may be used to attach portions of the panel to the neck part in such a manner that the panel 220 can be attached, detached, and re-attached at the outer edge as desired. A cable storage region 238 (FIG. 14) is defined within the wearable charger support 200 between the neck part 206 and the cable holder panel 220, and between the cable holder fasteners 222 and the panel inner edge 224. Storing a cable in this manner prevents the cable from becoming entangled with hair or jewelry and rubbing against the IMD recipient's neck. The cable holder panel 220 may be formed from any suitable material and, in some instances, may be loop material with the loops facing outward so that the cable holder 208 visually matches the chest and neck parts 202-206.

In the illustrated embodiment, where the neck part fastener 218 is the loop portion of a hook and loop fastener, the cable holder fasteners 222 may be hook portions of a hook and loop fastener. In other implementations, the cable holder panel 220 may be attached to the neck part 206, and detached therefrom, at the outer edge 236 through the use of snaps, buttons and any other suitable fasteners that are configured to mate with one another. A single elongate strip of hook material that extends along outer edge 226 may also be employed.

The first and second chest parts 202 and 204 may be connected to one another across the recipient's chest by way of a connector 240 in the manner illustrated in FIG. 1. Turning to FIG. 15, the exemplary connector 240 includes first and second straps 242 and 244 with respective fasteners 246 and 248 on the straps. The fasteners 246 and 248 include respective sets of projections 250 and 252. A projection 252 may be placed between the two of the projections 250 to secure the straps 242 and 244 to one another and to set the distance between the first and second chest parts 202 and 204. The projections 250 and 252 may be magnets, with the N-S directions oriented appropriately, or the projections 250 (or 252) may be magnets while the projections 252 (or 250) may be ferromagnetic. In other implementations, snaps, buckles, hook and loop fasteners or any other suitable instrumentality may be employed in place of the fasteners 250 and 252.

Another exemplary wearable charger support is generally represented by reference numeral 200a in FIG. 16. Wearable charger support 200a is essentially identical to wearable charger support 200 and similar elements are represented by similar reference numerals. For example, the wearable charger support 200a includes a first chest part 202, a second chest part 204, a neck part 206, a cable holder 208, and a connector 240. Here, however, an adjustable waist belt 254 that extends around the recipient's back is provided to hold the chest parts 202 and 204 against the recipient's chest. The exemplary waist belt 254 includes waist straps 256 and 258, which are secured to the chest parts 202 and 204, as well as fasteners 260 and 262 that are configured to mate with one another to secure the straps to one another. The fasteners 260 and 262 in the illustrated embodiment are respective hook and loop portions of a hook and loop fastener. In other implementations, snaps, buckles, magnets or any other suitable instrumentality may be employed in place of the fasteners 260 and 262. One or both of the waist belt straps 256 and 258 may include an extender so that the length may be varied as necessary.

The exemplary wearable charger support 200b illustrated in FIG. 17 is essentially identical to wearable charger support 200 and similar elements are represented by similar reference numerals. For example, the wearable charger support 200b includes a first chest part 202, a second chest part 204, a neck part 206 (FIG. 14), and a cable holder 208. Here, however, an antenna 264 (e.g., a Bluetooth antenna) may be provided. The antenna 264 may be located within the first chest part 202 (as shown), the second chest part 204, and/or the neck part 206.

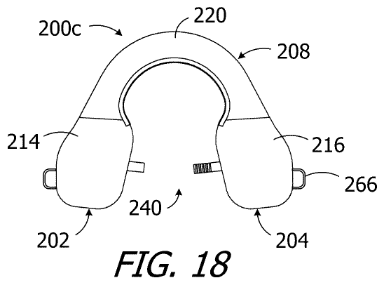

The exemplary wearable charger support 200c illustrated in FIG. 18 is essentially identical to wearable charger support 200 and similar elements are represented by similar reference numerals. For example, the wearable charger support 200c includes a first chest part 202, a second chest part 204, a neck part 206 (FIG. 14), and a cable holder 208. Here, however, the chest parts 202 and 204 each include a belt loop 266 through which a separate belt (not shown) may be threaded. The belt may extend through the belt loops 266 and around the recipient's back and chest to hold the chest parts 202 and 204 against the recipient's chest.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions. The inventions include any and all combinations of the elements from the various embodiments disclosed in the specification. The scope of the present inventions is limited solely by the claims set forth below.

1. An implantable medical device (IMD) charger apparatus for use with an IMD that is implanted in a recipient, the IMD charger apparatus comprising:
an IMD charger including
a transmission component having a transmission component housing, a primary coil within the transmission component housing, and a transmission component fastener associated with the transmission component housing,
an electronics component having an electronics component housing, a power source and a controller within the electronics component housing, and an electronics component fastener associated with the electronics component housing, and
a cable that extends from the transmission component to the electronics component; and
a wearable IMD charger support including
a first chest part with a first chest part fastener that is configured to mate with the transmission component fastener and/or the electronics component fastener,
a second chest part with a second chest part fastener that is configured to mate with the transmission component fastener and/or the electronics component fastener,
a neck part that extends from the first chest part to the second chest part, and
a cable holder associated with the neck part.

2. The IMD charger apparatus claimed in claim 1, wherein
the transmission component fastener and the electronics component fastener each comprise one portion of a hook and loop fastener.
the first chest portion fastener and the second chest portion fastener each comprise the other portion of a hook and loop fastener.

3. The IMD charger apparatus claimed in claim 1, wherein
the transmission component fastener and the electronics component fastener each comprise a hook portion of a hook and loop fastener.
the first chest portion fastener and the second chest portion fastener each comprise a loop portion of a hook and loop fastener.

4. The IMD charger apparatus claimed in claim 1, wherein
the transmission component housing defines a bottom and the transmission component fastener is located on the bottom of the transmission component housing; and
the electronics component housing defines a bottom and the electronics component fastener is located on the bottom of the electronics component housing.

5. The IMD charger apparatus claimed in claim 1, wherein
the cable defines a length that facilitates placement of the cable behind the neck when the transmission component and the electronics component on opposite sides of the recipient's chest.

6. The IMD charger apparatus claimed in claim 1, wherein
the length of the cable is about 10 inches to 30 about inches.

7. The IMD charger apparatus claimed in claim 1, wherein
the cable holder comprises a flexible panel that is movable between a closed position, where the flexible panel covers at least a portion of the neck part of the wearable IMD charger support, and an open position.

8. The IMD charger apparatus claimed in claim 7, wherein
the flexible panel defines a first edge that is permanently secured to the neck part and a second edge that is not permanently secured to the neck part.

9. The IMD charger apparatus claimed in claim 8, wherein
the neck part includes a neck part fastener; and
the cable holder flexible panel includes at least one panel fastener associated with the second edge that is configured to mate with the neck part fastener.

10. The IMD charger apparatus claimed in claim 9, wherein
the neck part fastener comprises a loop portion of a hook and loop fastener; and
the at least one panel fastener comprises at least one hook portion of a hook and loop fastener.

11. The IMD charger apparatus claimed in claim 9, wherein
the neck part fastener comprises a loop portion of a hook and loop fastener; and
the at least one panel fastener comprises a plurality of spaced hook portions of a hook and loop fastener.

12. The IMD charger apparatus claimed in claim 1, wherein
the first chest part, the second chest part and the neck part include a top fabric panel that forms the loop portion of a hook and loop fastener and a bottom panel formed from neoprene spandex.

13. A wearable implantable medical device (IMD) charger support for use with an IMD charger that includes a transmission component with a transmission component fastener, an electronics component with an electronics component fastener and a cable that extends from the transmission component to the electronics component, the wearable IMD charger support comprising:
a first chest part with a first chest part fastener that is configured to mate with the transmission component fastener and/or the electronics component fastener,
a second chest part with a second chest part fastener that is configured to mate with the transmission component fastener and/or the electronics component fastener,
a neck part that extends from the first chest part to the second chest part, and
means for covering the cable and holding the cable against the neck part.

14. The wearable IMD charger support claimed in claim 13, wherein
the first chest portion fastener and the second chest portion fastener each comprise one portion of a hook and loop fastener.

15. The wearable IMD charger support claimed in claim 13, wherein
the first chest portion fastener and the second chest portion fastener each comprise a loop portion of a hook and loop fastener.

16. The wearable IMD charger support claimed in claim 13, wherein
the neck part includes a neck part fastener.

17. The wearable IMD charger support claimed in claim 16, wherein the neck part fastener comprises a loop portion of a hook and loop fastener.

18. The wearable IMD charger support claimed in claim 13, wherein
the first chest part, the second chest part and the neck part include a top fabric panel that forms the loop portion of a hook and loop fastener and a bottom panel formed from neoprene spandex.

19. A wearable implantable medical device (IMD) charger support for use with an IMD charger that includes a transmission component with a transmission component fastener, an electronics component with an electronics component fastener and a cable that extends from the transmission component to the electronics component, the wearable IMD charger support comprising:
a first chest part with a first chest part fastener that is configured to mate with the transmission component fastener and/or the electronics component fastener,
a second chest part with a second chest part fastener that is configured to mate with the transmission component fastener and/or the electronics component fastener,
a neck part that extends from the first chest part to the second chest part and includes a neck part fastener, and
a cable holder including a flexible panel, with a first edge that is permanently secured to the neck part and a second edge that is not permanently secured to the neck part, and at least one panel fastener associated with the second edge that is configured to mate with the neck part fastener.

20. The wearable IMD charger support claimed in claim 19, wherein
the neck part fastener comprises a loop portion of a hook and loop fastener; and
the at least one panel fastener comprises at least one hook portion of a hook and loop fastener.

21. The wearable IMD charger support claimed in claim 19, wherein
the neck part fastener comprises a loop portion of a hook and loop fastener; and
the at least one panel fastener comprises a plurality of spaced hook portions of a hook and loop fastener.

22. The wearable IMD charger support claimed in claim 19, wherein
the first chest portion fastener and the second chest portion fastener each comprise a loop portion of a hook and loop fastener.

23. The wearable IMD charger support claimed in claim 19, wherein
the cable holder flexible panel comprises a fabric panel that has a front side and a rear side;
the front side of the cable holder fabric panel is a loop portion of a hook and loop fastener; and
the at least one panel fastener is located on the rear side of the cable holder fabric panel.

24. The wearable IMD charger support claimed in claim 19, wherein
the first chest, the second chest part and the neck part include a top fabric panel that forms the loop portion of a hook and loop fastener and a bottom panel formed from neoprene spandex.
